(19) United States
(12) Patent Application Publication
(10) Pub. No.: US 2018/0185675 A1
(43) Pub. Date: Jul. 5, 2018

(54) DEVICE AND METHODS FOR TOPICAL APPLICATION OF FLUIDS TO SKIN

(71) Applicant: NSE PRODUCTS, INC., Provo, UT (US)

(72) Inventors: Dale G. Kern, Hyde Park, UT (US); Richard R. Best, Mapleton, UT (US)

(21) Appl. No.: 15/842,077

(22) Filed: Dec. 14, 2017

Related U.S. Application Data
(60) Provisional application No. 62/434,931, filed on Dec. 15, 2016.

(51) Int. Cl. A61Q 17/04; B05B 11/00; B05B 1/16; A45D 24/24; A45D 34/04; B05C 17/035

(52) U.S. Cl. CPC A61Q 17/04; B05B 11/0072; B05B 1/169; A61K 9/0017; B05B 11/30; A45D 34/041; B05C 17/0357; A45D 24/24

(57) ABSTRACT

Devices and methods are disclosed herein apply a fluid to the skin of a human or animal, in particular skin bearing hair or fur. The device comprises an array of hair plow elements, each hair plow element comprising a skin contact surface. Device is adapted to apply a fluid to skin and simultaneously lift any hair or fur covering the skin or hair or fur sprouting from the skin away from the skin so that the fluid is applied preferentially to the skin rather than to any hair or fur covering the skin. Additional embodiments of the device are described comprising a transcutaneous electrical stimulator adapted for supplying a current to the fluid and the skin. Methods of use of the device are also disclosed.

### CROSS-REFERENCE TO RELATED APPLICATIONS

This application claims the benefit of U.S. Provisional Application No. 62/434,931 filed on Dec. 15, 2016.

### TECHNICAL FIELD

The invention is a device for applying a topical liquid, particularly liquid nutraceutical or cosmetic preparations, to skin that bears hair.

### BACKGROUND

Skin is the largest organ of the human body (also many mammals), with several important functions, including forming a physical barrier to the environment, protection against micro-organisms, allowing and limiting the inward and outward passage of water and electrolytes, ultraviolent radiation and toxic agents. Different kinds of topical liquid preparation exist for skin such as nutraceutical preparations, anti-aging or other care products, cosmetic products, and medicinal preparations. Some such products are applied to skin bearing little or no hair or fur. However, some liquid products require application to skin that bears hair or fur thereon and are designed for skin contact. Challenges exist in the application of such preparations to the skin or scalp rather than to the hair or fur sprouting therefrom or lying thereover. The simple addition of liquid preparations in bulk to the hair or fur is poorly targeted to the skin or scalp—much liquid may be soaked up by the hair or fur, for example by capillary action, wherein the liquid coats the surface of strands of the hair. Such “split application” of a topical preparation designed for application to scalp or skin is disadvantageous, because it is wasteful of the topical preparation—a significant proportion thereof does not find its way to the scalp or skin surface for which it is designed, but is absorbed by, adsorbed by, or coats the hair. Further, if the preparation is specifically designed for the scalp or skin, the application thereof to hair or fur may even be counterproductive to the health thereof.

The application of liquid treatments for scalp and hair/fur-bearing skin may use a simple nozzle in fluid communication with a containment holding the liquid treatment. Often, the liquid is dispensed by application of increased pressure to the liquid, which forces liquid through the nozzle. Such application is slow, requiring separation of hair or fur with one hand and application from a bottle or containment with the other. A single nozzle furnishes the liquid at a low rate of area coverage of skin, and such application therefore requires time. Further, separation or displacement of individual strands of hair is time-consuming and requires dexterity. Much of the liquid may nevertheless be lost to application onto hair strands.

There is an ongoing need for devices for the topical application of liquid preparations to hair- and fur-bearing skin, wherein application of the liquids is targeted preferentially to the skin or scalp and covers more than a single spot or line in a single sweep.

### SUMMARY

Disclosed herein is a device for application of a fluid to a skin surface with hair covering, the device comprising a hollow manifold body surrounding and defining a chamber therein and comprising an input end and an output end; and a plurality of hair plow elements attached to and extending away from the output end of the manifold body and arranged in a generally linear array with a spacing interval between adjacent hair plow elements, each hair plow element comprising a skin contact surface and a hollow stem extending from the skin contact surface toward the manifold body, the stem connecting the element to the output end, each hollow stem surrounding and defining a fluid delivery conduit within the stem, the conduit being in fluid communication with the chamber, the skin contact surface comprising a leading edge comprising a plow tip, the skin contact surface defining a fluid delivery orifice with a fluid delivery element positioned in the orifice and disposed within the conduit, and adjacent stems defining a hair flow passage therebetween. In embodiments, each hair flow passage of the device is arcuate in an aspect perpendicular to adjacent stems and to the skin contact surfaces, and in the aspect the distance between adjacent stems is minimum proximal to the skin contact surfaces. The device of claim 1, wherein the hair flow passage is generally keyhole-shaped in an aspect perpendicular to the stems and the skin contact surfaces. In embodiments, the shape of each skin contact surface is approximately lenticular. In embodiments, the plow tips are rounded off. In embodiments, each skin contact surface of the device comprises a trailing edge and tapers outward from the leading edge around the delivery orifice and then inward toward the trailing edge. In embodiments, the fluid delivery element is selected from: (a) a wicking element in fluid communication with the chamber and extending to the delivery orifice; or (b) a roller ball captured in the delivery orifice and in fluid communication with the chamber. In embodiments, the device further comprises a liquid cosmetic and/or nutraceutical preparation disposed within the fluid delivery conduit and the chamber. In embodiments, the fluid delivery element is effective for delivering a fluid with a water-like viscosity and the fluid is a cosmetic and/or a nutraceutical preparation for the skin. In some embodiments, the device further comprises a hollow fluid supply element attached to the input end of the body, the hollow fluid supply element defining therein a fluid reservoir in fluid communication with the chamber, wherein the fluid supply element is selected from a hose, a fixed container, or a removable container.

In some embodiments, the device further comprises a source of electrical current and a plurality of contact electrodes in an array, wherein each skin contact surface of a hair plow element comprises an contact electrode in electrical contact with the source.

Also disclosed herein is a method of application of a fluid to skin bearing hair, the method comprising applying the skin contact surfaces of the device to a first area of skin and/or hair of a human or animal subject; moving the device in one of either of the directions of plow tips of the device for a first period of time while maintaining contact between the skin contact surfaces and a second area of the skin and/or hair; and removing the skin contact surfaces from the skin and/or hair, wherein at least some of the fluid is deposited as a layer on some or all of the second area of the skin and/or hair.

### BRIEF DESCRIPTION OF THE DRAWINGS

FIG. 1 is as isometric view of one of the embodiments of an applicator device.

FIG. 2 is a view of the embodiment of the device of FIG. 1 taken generally perpendicular to the surfaces of fluid delivery elements.

FIG. 3 is a cut-away schematic view of the device of the first embodiment shown in FIG. 1 depicting internal aspects.

FIG. 4 is a cross-section view of another embodiment of the device, the embodiment having a wicking element serving as fluid delivery element and the embodiment comprising a removable container as fluid supply element.

FIG. 5 is a cross-section view of another embodiment of the device, the embodiment comprising roller balls and wicking elements in combination therewith and the device comprising a cap with a pressure-equalization valve.

FIG. 6 is a view of the device being moved in the direction of plow tips of fluid delivery elements, showing how the device gathers hair into bunches disposed within hair flow passages.

FIG. 7 is a schematic of an embodiment of the device comprising a common electrode used for galvanic effects.

FIGS. 8-11 show some possible shapes for the skin contact surfaces of fluid delivery elements.

FIG. 12 is a schematic view of another embodiment with electrodes used for galvanic effects.

FIGS. 13A-13C are schematic views of hair plow elements in three temporal stages in sequence of (a), (b), and (c) of movement of the device in a direction of the plow tips along a skin surface bearing hair with skin contact surfaces applied to skin and/or hair.

FIGS. 14A-14C are schematic side views of hair plow elements in three temporal stages in sequence of (a), (b), and (c) of movement of the device in a direction of the plow tips along a skin surface bearing hair with skin contact surfaces applied to skin and/or hair.

FIGS. 15A-15C are schematic views of hair plow elements in a cross-section with three temporal stages in sequence of (a), (b), and (c) of movement of the device in a direction of the plow tips along a skin surface bearing hair with skin contact surfaces applied to skin and/or hair.

FIG. 16 is an isometric view of one embodiment of the device that includes a removable container as fluid supply element and a handle as part of the body.

### BRIEF DESCRIPTION OF THE INVENTION

### Detailed Description

Although the present disclosure provides references to preferred embodiments, persons skilled in the art will recognize that changes may be made in form and detail without departing from the spirit and scope of the claims. Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the multiple possible embodiments for the appended claims.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

As used herein, the terms “comprise(s),” “include(s),” “having,” “has,” “can,” “contain(s),” and variants thereof are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the term “optional” or “optionally” means that the subsequently described event or circumstance may but need not occur, and that the description includes instances where the event or circumstance occurs and instances in which it does not.

As used herein, the term “about” modifying, for example, a dimension, a period of time, a volume, a pressure, a coefficient of friction, a quantity of an ingredient in a composition, a current intensity or pulse width, and like values, and ranges thereof, employed in describing the embodiments of the disclosure, refers to variation in the numerical quantity that can occur, for example, through typical measuring procedures; through inadvertent error in these procedures; through differences in the manufacture, source, or purity of starting materials or ingredients used to carry out the methods, and like proximate considerations. Where modified by the term “about” the claims appended hereto include equivalents to these quantities. Further, in all embodiments where “about” is employed to describe any range of values, for example “about 1 to 5” the recitation means “about 1 to about 5” and/or “1 to about 5” and/or “about 1 to 5” and/or “1 to 5” unless specifically limited by context.

As used herein, the word “substantially” modifying, for example, the type or quantity of an ingredient in a composition, a property, a measurable quantity, a method, a position, a value, or a range, employed in describing the embodiments of the disclosure, refers to a variation that does not affect the overall recited composition, property, quantity, method, position, value, or range thereof in a manner that negates an intended composition, property, quantity, method, position, value, or range. Intended properties include, solely by way of nonlimiting examples thereof, elasticity, modulus, hardness, and shape; intended positions include position of a first treatment feature relative to a second treatment feature. Where modified by the term “substantially” the claims appended hereto include equivalents to these types and amounts of materials.

As used herein, the term “animal” refers to mammals but excludes humans.

As used herein, the term “user” means a human who utilizes the devices, apparatuses, or methods of the invention on a human or animal subject. In some embodiments the user is also the human subject. Unless otherwise specified, the term “user” includes embodiments wherein the user utilizing the devices or methods of the invention is not the subject, and wherein the user utilizing the devices or methods of the invention is the human subject.

As used herein “subject” means a human or animal to whose skin a fluid is applied by means of the device of the invention.

As used herein, “water-like viscosity” means a dynamic viscosity of about 0.3 cP to about 5 cP measured in a range of 15 to 40° C.

As used herein, the term “skin” unless otherwise specified means mammalian skin, animal or human, and includes the scalp of the human head.

As used herein, unless otherwise specified the term “hair” includes human hair, animal hair and animal fur.

As used herein, the term “lenticular shape” refers to a range of shapes resembling or approximating to that of a biconvex lens in cross-section. It is construed herein to include the elliptical shapes; the shape vesica piscis, the shape that is the intersection of two circles with the same radius, the circles intersecting such that the center of each circle lies on the perimeter of the other; the shape of a lemon in cross-section along the long axis; and the like.

As used herein, the term “stadium” or “obround” is used to refer to a range of shapes resembling or approximating to the shape of the perimeter of a rectangle with two semicircles attached thereto, each semicircle having a diameter of the same length as the short side of the rectangle, and each semicircle being attached to one short side of the rectangle, the ends of the semicircle being attached to the ends of the short side. The term “obround” includes the range of shapes resembling or approximating to the shape of the perimeter of a rectangle with two identical arcs attached thereto, the straight-line distance between the ends of each arc having the same length as the short side of the rectangle, and each arc being attached to one short side of the different rectangle.

### DISCUSSION

### First Embodiments

Handheld applicators for liquid topical applications for skin are a commonplace. In many cases such applicators are little more than a plastic dispenser such as a bottle or other container with a single nozzle defining a single orifice: the user squeezes the applicator, thus raising the pressure of the liquid contents thereof above atmospheric pressure. The pressure differential between the liquid within the dispenser and ambient air pressure causes the liquid contents to be expelled from the nozzle. The user holding the dispenser contacts the nozzle against the skin, keeping the nozzle either against or in very close proximity to the skin. The user moves the dispenser across the skin, thereby laying a line of the topical liquid thereon. Such an application covers a low proportion of the area of the skin and is reliant on the properties of the liquid to preferentially adhere to and wet out the skin as opposed to any hair lying thereon and/or sprouting therefrom.

Even if multiple nozzles were supplied for such a dispenser, only marginal improvements in the application rate per unit area of skin are made. In order to cover an advantageously large proportion of the area of the skin surface, the liquid contents dispensed from the nozzle would still need to be designed to spread over the surface of the skin in preference to wicking into the hair or fur covering the skin and growing therefrom. Further, the problem exists of how to displace hair or fur and lift it away from the skin around each nozzle, in order that the topical preparation be applied to the skin or scalp and not to the hair or fur.

Some applicators comprise a plastic bottle having an orifice in one end with an applicator ball disposed therein in fluid communication with a liquid preparation. The user holds the applicator in an inverted or partially inverted position, and gravity supplies the liquid contents to the surface of the applicator ball disposed within the applicator. Rolling the ball over the surface of the skin and hair sprouting therefrom exposes applicator ball surface coated with the liquid preparation—the preparation contacts the skin and hair growing therefrom, and is transferred at least in part thereto. Such “single-ball applicators” are used, for example, to apply antiperspirants and deodorants to the skin under the arms. The preparation is applied to skin and hair without a great deal of selectivity, but a particularly high proportion of the applied topical liquid is not lost to the hair under the arms because these skin locations typically bear a low density of hair. In order to provide a conveniently high rate of liquid preparation to the underarm skin surface, the ball is made large and the applicator applies the antiperspirant and deodorant without distinction over the underarm skin and hair. These devices are unsuitable for use with skin such as a scalp bearing thick hair or skin bearing fur, because such applicators simply apply the contents thereof to the hair or fur lying over the skin—the hair lies en masse between the applicator ball and the skin surface.

A further type of applicator used for dispensing antiperspirant and/or deodorant comprises a rounded off applicator surface defining a plurality of nozzles. Such applicators are supplied with a means for pressurizing the liquid or gel contents. One example of such means is a piston or plunger. The user turns a screw which applies force to the piston or plunger, and the latter is pushed so that it applies pressure to the liquid or gel contents. Like single-ball applicators, such “multi-nozzle applicators” are unsuitable for applying a liquid or a gel to a skin surface bearing thick hair. Rather, the use of such applicators on such skin bearing thick hair simply results in application of the preparation to the hair, because the hair lies en masse between the applicator nozzles and the skin surface.

Like applicators exist for the application of topical preparations to animals having fur. Some flea and tick preparations are administered by disposable single-nozzle dispensers. Many such products are administered monthly over the summer months to protect the animal from infestation. The user must manually part the hair or fur, e.g. between the shoulder blades, and squeezing the applicator applies its liquid contents to a single spot.

Nutraceutical, cosmetic, medicinal, and other liquid and/or gel preparations designed for topical application to the human scalp or skin bearing hair include oils, creams, liquids, and/or gel preparations for nourishing the skin; cosmetic applications; or treating itching, psoriasis, fungal infections, dermatitis, dry skin, eczema, scalp folliculitis, scalp acne, sunburn, or skin aging.

Some applicators and systems incorporating them are designed to apply liquids to the skin in association with a galvanic current such as NU SKIN® Galvanic Spa® System II, available from NU SKIN® ® Enterprises of Provo, Utah.

The devices described herein solve the problem of applying liquid nutraceutical or medicinal preparations to human scalp or skin bearing thick and/or long hair, or animal skin bearing hair or fur, wherein the device of the invention, lifting hair or fur away from the skin, applies a liquid preparation thereto.

In embodiments, the devices comprise galvanic attachments, enabling the topical application of nutraceutical liquids to the skin, followed or accompanied by galvanic treatment.

In embodiments, there is provided a device for the application of a fluid to a skin surface with hair covering, the device comprising a hollow manifold body surrounding and defining a chamber therein and comprising an input end and an output end. The device comprises a plurality of hair plow elements attached to and extending away from the output end of the manifold body and arranged in a generally linear array with a spacing interval between adjacent hair plow elements, each hair plow element comprising a skin contact surface and a stem extending from the skin contact surface toward the manifold body, the stem connecting the element to the output end. Each stem is hollow, defining a fluid delivery conduit within the stem. Each skin contact surface comprises a leading edge comprising a plow tip. Each skin contact surface defines a fluid delivery orifice with a fluid delivery element positioned in the orifice and disposed within the fluid delivery conduit, adjacent stems defining a hair flow passage therebetween.

In embodiments, the hollow manifold body is substantially a shell, that is, the hollow interior occupies from about 75.0% to about 99.5% of the volume of the body. In embodiments, the hollow body is partially hollow, defining a hollow chamber capable of holding a fluid therein, wherein the chamber occupies from about 50% to about 75% of the volume of the body, in embodiments from about 25% to about 50%, in embodiments from about 5% to about 25%. The body comprises an input end and an output end with a plurality of hair plow elements extending therefrom. In embodiments, the body in cross-section at the input end has a shape selected from obround, ellipse, oval, and a circle. In embodiments, the body in cross-section is has an obround shape. The body furnishes fluid communication between the input end and the output end. In various embodiments, fluid enters the manifold body at the input end; and when the device is in use, fluid flows in a general direction from the input end to the output end.

Each hair plow element comprises a skin contact surface and a hollow stem, each hollow stem extending from each skin contact surface to the manifold body. Each stem has an axis extending from the hair plow surface to the manifold body, wherein the stem is symmetrical about the axis. In embodiments, the axes are parallel to each other.

In embodiments, the hair plow elements are arranged in a generally linear array, i.e. the axes are parallel and the skin contact surfaces viewed in an aspect in the direction of the axes are arranged in a straight line. In other embodiments, the hair plow elements are arranged in an arc when viewed in an aspect in the direction of the axes. In embodiments the hair plow elements of the plurality of hair plow elements are spaced at regular intervals having a spacing interval. The spacing interval is the distance between the axis of one stem to the axis of an adjacent stem. In embodiments, the spacing interval is approximately 10 mm.

In embodiments, the fluid delivery element is selected from (a) a wicking element in fluid communication with the chamber and extending to the delivery orifice; or (b) a roller ball captured in the delivery orifice and in fluid communication with the chamber. The wicking element is elongated in the direction of the hollow stem and is disposed within the fluid delivery conduit. Each fluid delivery conduit connects a fluid delivery orifice to the chamber and thereby furnishes fluid communication between each fluid delivery orifice and the chamber. In embodiments, a wicking element in fluid communication with the chamber is disposed within the fluid delivery conduit. In some embodiments, the wicking element is a fluid delivery element and supplies fluid by capillary action to the fluid delivery orifice. In some such embodiments, the wicking element is tubular in shape and fills the fluid delivery orifice—when a user presses the skin contact surfaces against a skin, each wicking element contacts the skin and supplies fluid to the skin as the device and the skin contact surfaces thereof are moved along the skin in the direction of the plow tips.

In other embodiments, the fluid delivery element is a roller ball captured within the fluid delivery orifice. The roller ball has a diameter greater than at least one dimension of the fluid delivery orifice, such that the roller ball is captured within the fluid delivery orifice and the fluid delivery conduit. In embodiments, the fluid delivery orifice is circular and the diameter of the roller ball is greater than the diameter of the orifice. In such embodiments, the roller ball is disposed within the fluid delivery conduit proximal to the skin contact surface, and the wicking element is in fluid contact with the chamber and in contact with the roller ball. Disposed within the fluid delivery conduit is means for urging the roller ball into the fluid dispersion orifice and means for urging the wicking element into contact with the roller ball. When a fluid is disposed within the chamber, the wicking element fills with fluid. In embodiments, the means for urging the roller ball into the fluid dispersion orifice comprises, consists of, or consists essentially of a spring. A user urges the skin contact surfaces toward a skin surface in an action causing the roller balls to be pressed into the fluid delivery orifices against the means for urging the roller balls, the action also causing the skin contact surfaces to contact the skin—part of the surface of the roller balls remains in contact with the skin. When a user moves the device in the direction of the plow tips and maintains contact between the skin contact surfaces of the device and the skin, the roller balls rotate, and the wicking elements deposit a film of fluid on the surfaces of the roller balls as each surface of a roller ball passes through an area of contact between the roller ball and its associated wicking element within the associated fluid delivery conduit. As the roller balls rotate, the films of fluid on the surfaces of the roller balls become exposed in the fluid delivery orifices, and fluid deposits on the skin as a portion of the surface of each roller ball carrying fluid thereon contacts the skin. Fluid flows from the chamber, through the wicking elements, onto the roller balls and is applied onto the skin. A film of the fluid is applied to an area of the skin and collects between skin contact surfaces and the skin.

Each wicking element can be separate and in fluid communication with the chamber, or one or more or all wicking elements can join a manifold disposed within the body, the manifold being in fluid communication with the one or more wicking elements and with the chamber. In embodiments, therefore, the device comprises a manifold extending between one or more wicking elements and the chamber, the manifold being in fluid communication with the chamber and with the one or more wicking elements. In embodiments, the manifold extends between all the wicking elements disposed within the device and the chamber, and is in fluid communication between the wicking elements and the chamber. In embodiments, the manifold comprises the same material as the wicking elements.

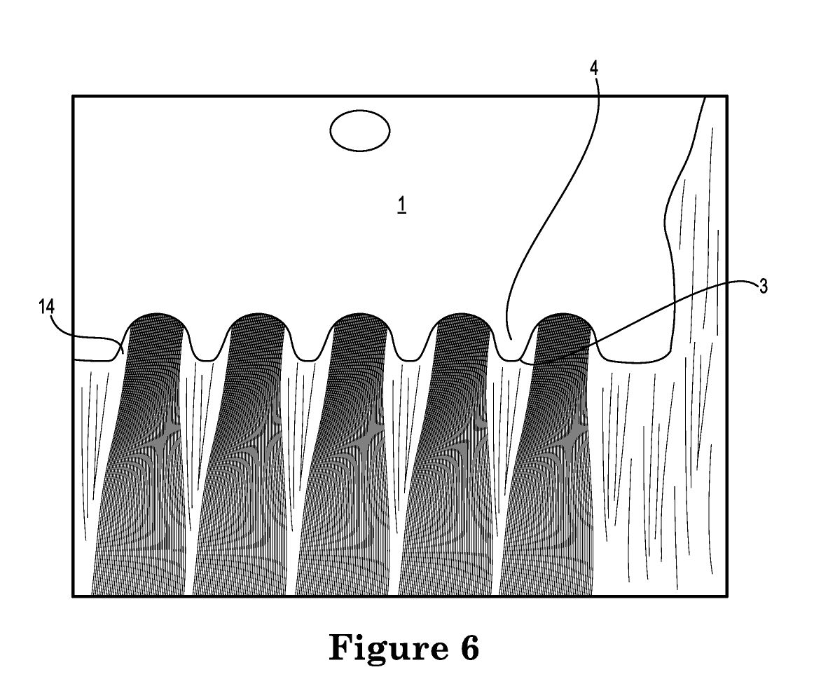

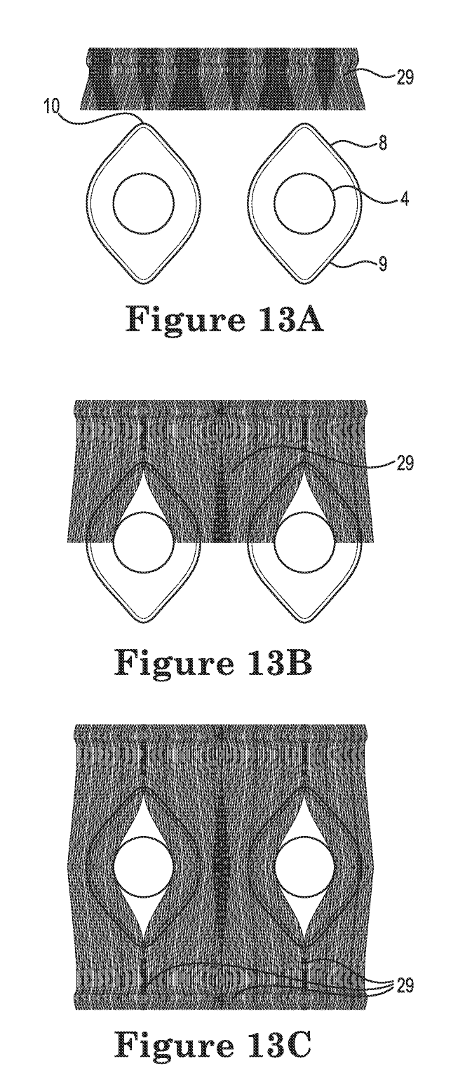

As the user thus moves the device in the direction of the plow tips with the skin contact surfaces in contact with the skin, hair covering the skin and sprouting therefrom is parted by the motion of the plow tips, is displaced either side thereof, is lifted over the foot of each stem, and is collected into a hair flow passage; and thereby is advantageously held away from the surface of the skin. FIG. 6 shows an image of hair collected thus into hair flow passages as a user moves the device. FIGS. 13A-C, 14A-C, and 15A-C show different aspects of the process.

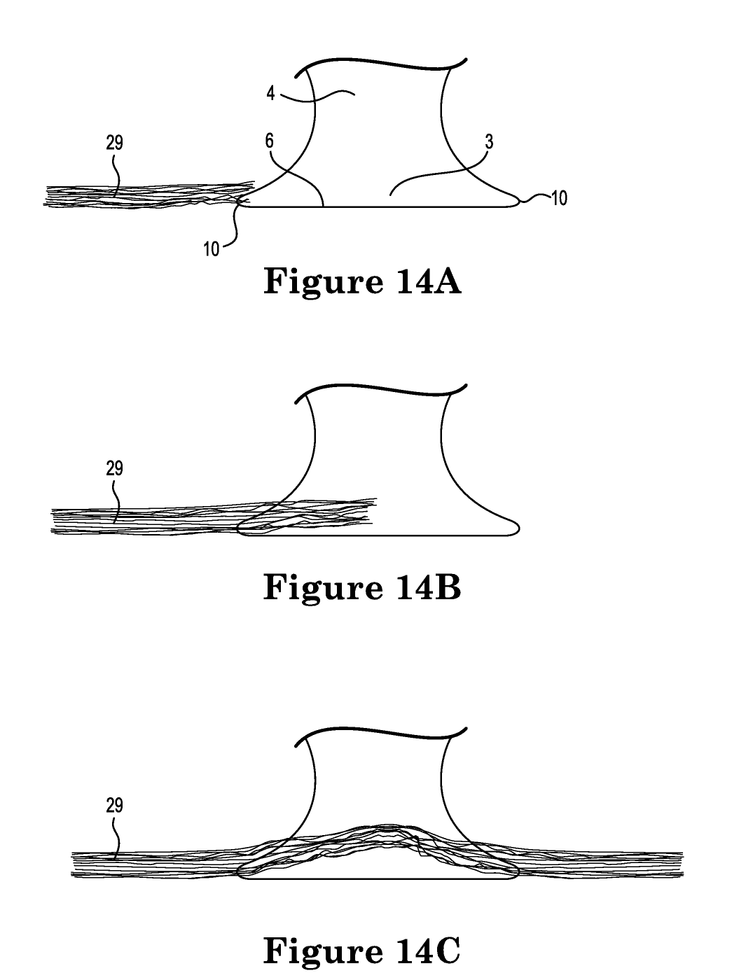

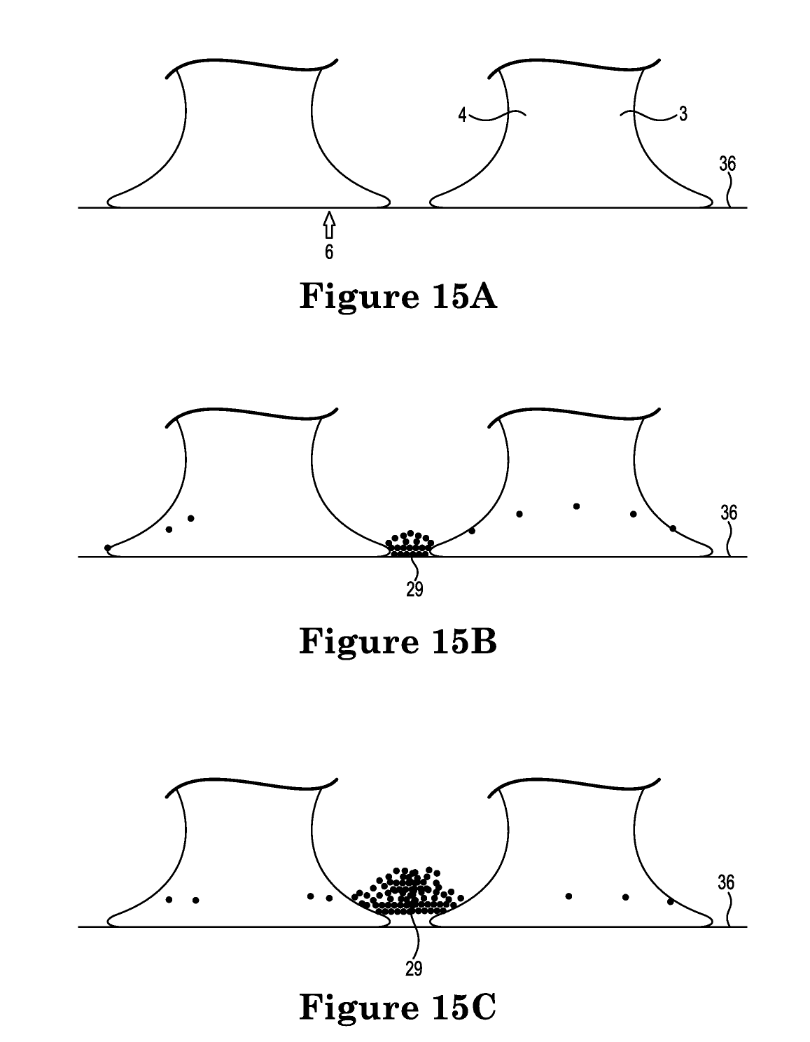

Each of FIGS. 13A-C, 14A-C, and 15A-C schematically depicts a sequence of three different temporal stages in the movement of hair plow elements through hair, in order (A), (B), and (C). With reference to FIGS. 13A-C, 14A-C, and 15A-C: The user (not shown) applies the skin contact surfaces 6 to an area of skin and/or hair adjacent to skin 36 covered with a layer of hair 29. If the hair lies in a particular direction in the first area of skin and/or hair, the user applies the skin contact surfaces 6 of the device to the first area of skin and/or hair such that plow tips 10 of the device are oriented in a direction pointing approximately along the direction in which the hair 29 lies. The user, maintaining contact between the skin contact surfaces and the skin and/or hair, moves the device in a motion substantially parallel to the surface of the skin 36 and in a direction of the plow tips across the surface of the skin and/or hair, i.e., approximately along the direction in which the hair lies, either towards the hair roots or away from the hair roots with respect to individual hairs. As the user moves the skin contact surfaces, hair strands collectively separate around stems 4 as shown in FIGS. 13B, 14B, and 15C and rides up over the wider, lower portions of the hair plow elements, gathering into bunches disposed in hair flow passages as represented in FIG. 15C.

Proximal to the skin contact surface, each hair plow element and the stem thereof tapers along the axis with increasing distance from the skin contact surface—the stem tapers in both dimensions perpendicular to the axis of the stem. Because the stem tapers in the two dimensions perpendicular to the axis of the stem, as the stem thus extends toward the skin contact surface it comprises a splayed foot, or “foot”. In some embodiments, the girth of each hair plow element and of the stem thereof reaches a minimum and then enlarges again as the stem approaches the manifold body. In embodiments, adjacent hair plow elements are closest together proximal to the skin contact surfaces: put differently, in such embodiments the distance between adjacent hair plow elements is least at or proximal to the skin contact surface.

In embodiments, each skin contact surface in the array of hair plow elements has the same shape and area. The skin contact surface comprises a leading edge and a trailing edge. The In some embodiments, the leading edge and the trailing edge each comprise a plow tip. In other embodiments, the leading edge comprises a plow tip and the trailing edge does not comprise a plow tip. In embodiments wherein both the leading edge and the trailing edge each comprise a plow tip, a user can apply a fluid to a surface of skin by applying the skin contact surfaces of a the device to a surface of skin bearing hair and moving the device in either of the two directions of a plow tip. The leading edge of a skin contact surface comprises two halves meeting at and extending from the plow tip and diverging from each other in the general direction of the trailing edge. In embodiments, the two halves are convex.

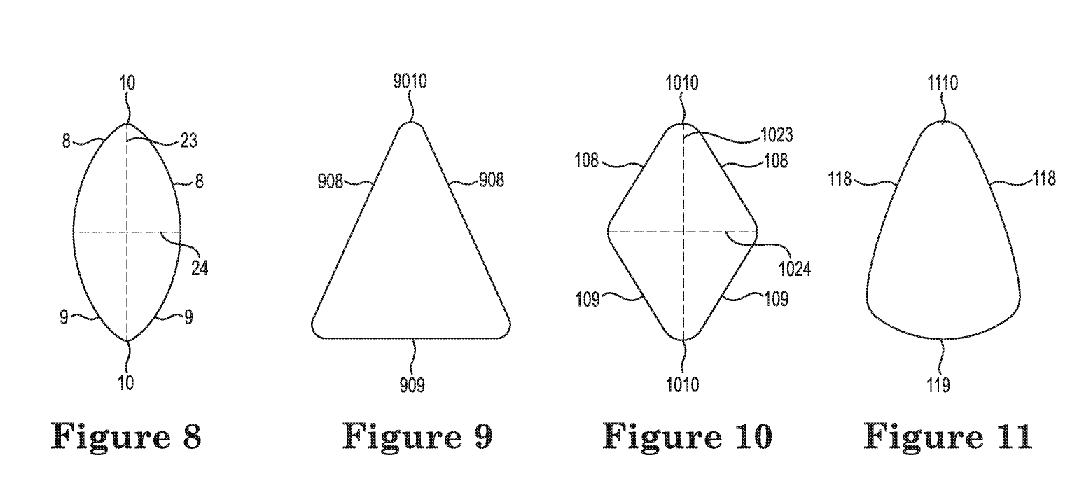

Exemplary embodiments of the shape of the skin contact surface are depicted in FIGS. 8-11. In embodiments, the shape of the skin contact surface is selected from approximately lenticular, approximately reuleaux triangle, or approximately pear shape. In embodiments wherein the shape of the skin contact surface is approximately lenticular, the lenticular shape has a major axis and a minor axis. The major axis extends from the plow tip of the leading edge to the plow tip of the trailing edge. The minor axis is perpendicular or approximately perpendicular to the major axis. In embodiments, the ratio of the width of the skin contact surface along the minor axis to the length of the skin contact surface along the major axis is from about 6:18 to 10:12, in embodiments from about 1:2 to 10:12, in embodiments 3:5 to 10:12, in embodiments about 7:10. In some such embodiments, the width of the skin contact surface along the minor axis is about 8.6 mm and the length of the skin contact surface along the major axis is about 12.1 mm. In some embodiments, each skin contact surface is approximately perpendicular to the axis of each stem; in others the skin contact surfaces are slanted towards the perpendicular. In embodiments, the skin contact surfaces lie within a single plane.

In embodiments, the plow tips are rounded off. A rounded off plow tip means that the plot tip does not terminate in a one dimensional point but that the tip is narrow but generally arcuate rather than pointed. Applicants have found that rounded off plow tips may be more comfortable for a subject along whose skin the device of the invention is moved in one direction of the plow tips and to whose skin the skin contact surfaces are applied. However, narrow plow tips may assist in lifting hair from skin during such motion.

In embodiments, the stems in a cross-section parallel to the skin contact surfaces have about the same shape as the skin contact surfaces but a lower area. The stems taper as they extend from the skin contact surfaces toward the manifold body. In embodiments, the stems taper as they extend from the skin contact surfaces toward the manifold body, then the stems enlarge until they join at the manifold body—in such embodiments, the closest distance between adjacent stems increases along the axes of the stems away from the skin contact surfaces, the distance reaching a maximum before decreasing again approaching the manifold body. Although the distance between adjacent hair plow elements is a minimum at or proximal to the skin contact surfaces thereof, there is a gap between adjacent skin contact surfaces. As is described infra, such a gap is necessary to allow hair flow passages to receive hair raised above the skin. Adjacent stem shapes define a hair flow passage between the adjacent stems. In embodiments, each hair flow passage is generally arcuate. In embodiments, when viewed in an aspect perpendicular to the stems and the array of hair plow elements (i.e. when the viewing direction is head on to the plow tips), the hair flow passage is keyhole-shaped, that is resembling an upper case omega (a), the feet thereof corresponding to part of the skin contact surfaces. Applicants have discovered that hair flow passages having such a keyhole-shape defined by stems and hair plow elements that taper in two dimensions perpendicular to the axes of the stems with increasing distance from the skin contact surfaces show advantages in applying fluid preferentially to skin. When a user moves an array of such hair plow elements in the direction of the plow tips with the skin contact surfaces substantially flat against a skin surface, as noted supra, hair covering the skin and growing therefrom is parted by the motion of the hair plow elements, is parted either side of each plow tip, and rides up over the foot gathering in bunches in the hair flow passages, as shown in FIG. 6. The hair is thus moved away from the skin by the motion of the device and the hair plow elements attached thereto, wherein the motion of the hair plow elements lifts and gathers hair into the hair flow passages. The hair is thus raised and held clear of the skin surface proximal to the skin contact surfaces. The same motion of the device, also applies fluid onto the skin.

In embodiments, the device further comprises a hollow fluid supply element attached to the input end of the body, wherein the fluid supply element defines a fluid reservoir, the fluid reservoir being in fluid communication with the chamber. In embodiments, the fluid supply element is selected from a hose, a fixed container, or removable container. In embodiments, the removable container is a refill, wherein the reservoir is filled with or partially filled with fluid. In embodiments, there is supplied an apparatus comprising, consisting of, or consisting of any of the embodiments of the device of the invention described herein and a refill, wherein the refill comprises a fluid and a containment therefor, wherein the containment is filled or partially filled with the fluid. In embodiments, the refill is a removable container adapted for attachment to the input end of the body of the device.

Embodiments of the invention will now be described in more detail with reference to the figures.

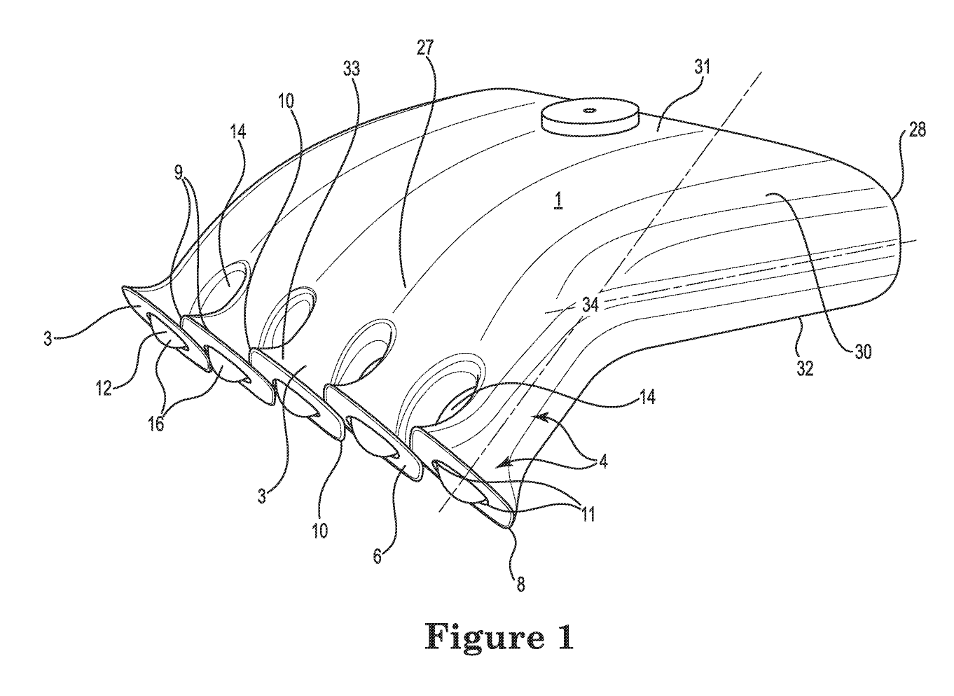

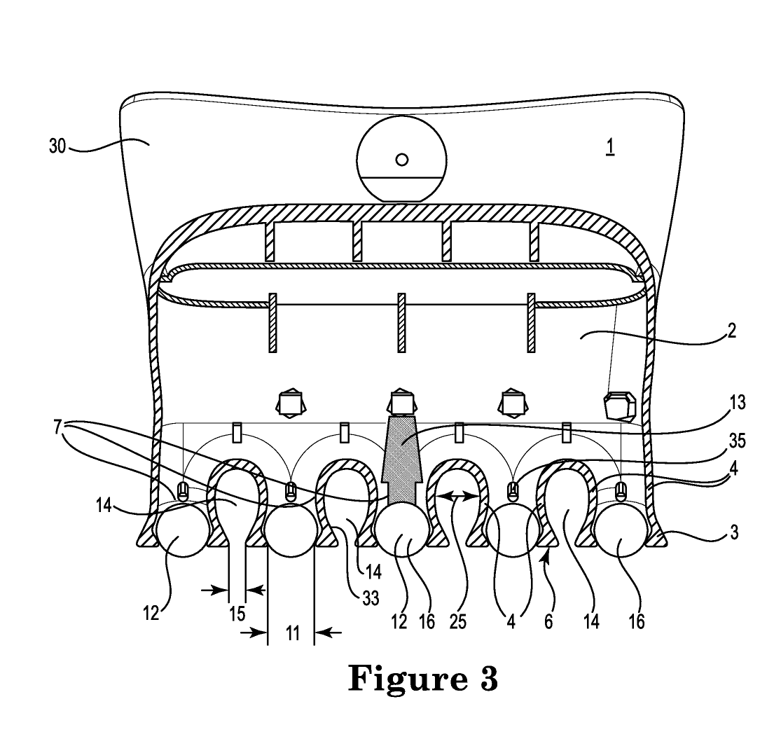

FIG. 1 shows one embodiment of the invention. With reference to FIG. 1; the device 1 comprises a body 30 comprising an input end 28 and an output end 27. The body 30 in cross-section at the input end has an obround shape. The body at input end 28 has a top surface 31 and a bottom surface 32. A plurality of hair plow elements 3 extends away from and is joined to the output end 27 of the body 30. Each hair plow element 3 has an associated stem 4 extending from the output end 27 of the manifold body. Each hair plow element terminates in a skin contact surface 6, the plurality of hair plow elements terminating in a plurality of skin contact surfaces, wherein the plurality of the contact surfaces lie substantially within a single plane. Each skin contact surface comprises a leading edge 8 and a trailing edge 9. Each of the leading edge and the trailing edge comprises a plow tip 10. Each skin contact surface has an associated fluid delivery orifice 11 defined therein. Each stem has an associated axis extending from the skin contact surface, wherein each skin contact surface is approximately perpendicular to each axis of the stem and each stem is generally symmetrical about the axis. The fluid delivery orifices 11 are circular in shape. Each stem tapers away from the skin contact surface toward the body, as shown in FIG. 1; and approaching the skin contact surface and in proximity thereto, the stem splays out in two dimensions into a foot 33. The stems and the hair plow elements that comprise them thus define arcuate hair flow passages 14 therebetween. In embodiments, hair flow passages have a generally keyhole shape, as depicted in FIG. 3. Other shapes are possible, with the proviso that gap 15 is less than distance 25. In embodiments, when viewed in an aspect perpendicular to the stems and the array of hair plow elements (i.e. when the viewing direction is head on to the plow tips), each hair flow passage is shaped like the Roman letter U with serif, wherein gap 15 is less than distance 25.

The body 30 extending from the input end 28 to the output end 27 in an aspect perpendicular to the axes of the stems and the plane of the skin contact surfaces, is bent or curved, such that in that aspect the axes of the stems 4 are at an angle with respect to the top surface of the body 31. This is indicated in FIG. 1 by intersecting construction lines 34 (not part of the device). In embodiments, a user holds the device by the body 30 with the user's hand, and the user lifts hair covering a skin surface and applies the array of skin contact surfaces to the skin surface so that the skin contact surfaces 6 are in contact with the skin (not shown). The user then releases the hair. The bend or curve indicated in FIG. 1 by intersecting lines 33 provides a comfortable angle for the user to hold the device while maintaining the plurality of skin contact surfaces 6 against the skin.

Disposed within each fluid delivery conduit shown in FIG. 1 is a roller ball 16. In the embodiment shown in FIGS. 1, 2, and 3, each fluid delivery element 12 is a roller ball 16 recessed into the stem. Also disposed within each fluid delivery conduit is a spring (not shown) urging each roller ball into each fluid delivery orifice so that part of each roller ball protrudes therefrom. The user applies a counterforce maintaining the skin contact surfaces against the skin, the counterforce opposing the collective forces of the springs urging the roller balls into the respective associated fluid delivery orifice, and said counterforce retractably impels the roller balls into their respective fluid delivery conduits, so that each roller ball contacts the skin substantially at a point lying roughly in the same plane as the skin contact surfaces.

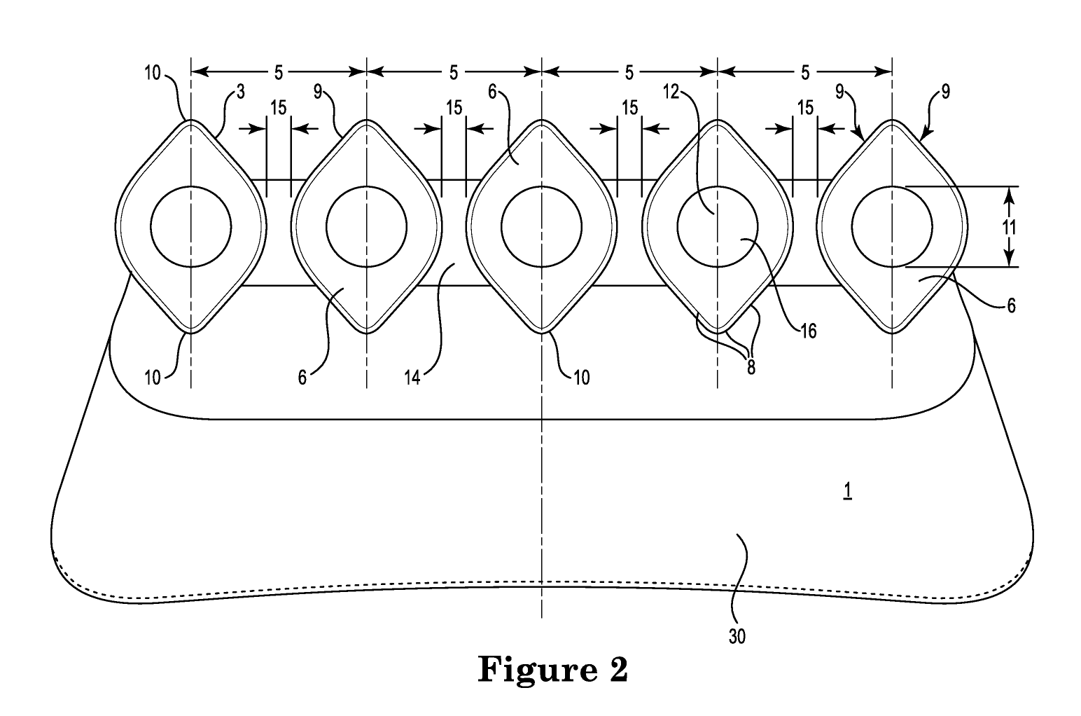

The same embodiment of the device as that exhibited in FIG. 1 is shown in a different aspect in FIG. 2. The skin contact surfaces 6 are disposed at regular intervals having a spacing interval 5. The skin contact surfaces define a gap 15, which is the closest distance between adjacent hair plow elements. Each of the skin contact surfaces comprises a leading edge 8 and a trailing edge 9 symmetrically disposed about the center of the skin contact surface 6. The leading edge and the trailing edge each comprise one plow tip 10. Each skin contact surface is approximately lenticular in shape and symmetrical about the axis of the stem. Each skin contact surface 6 defines a substantially circular fluid delivery orifice 11. Disposed within and captured by each dispersion orifice is a substantially spherical roller ball 16 associated therewith. Each roller ball has a diameter larger than that of the fluid delivery orifice associated therewith. Absent a pressure urging the roller balls 16 into the delivery orifices associated respectively therewith, each roller ball substantially fills each fluid delivery orifice. The roller balls are substantially free to rotate, and the roller balls rotate on application of a force tangent thereto.

FIG. 3 depicts another aspect of the embodiment of the device shown in FIG. 1 and FIG. 2. FIG. 3 depicts a cutaway to reveal fluid delivery conduits 7. Disposed within each fluid delivery conduit is a roller ball 16 and associated wicking element 13. For clarity, only one wicking element 13 is shown in FIG. 3. A portion of each wicking element 13 contacts a portion of the surface of the roller ball 16. In embodiments, the wicking element is shaped to conform to the surface of the roller ball, as shown schematically in FIG. 3. The wicking elements are in fluid communication with a chamber 2 holding the fluid for application. Associated with each roller ball and disposed within each stem 4 is means 35 for urging the roller ball into the fluid delivery orifice. The means for urging the roller ball may be a spring immovably affixed to the stem 4 at least one point or location of the stem and extending from the point or location to the roller ball, wherein the spring is compressed in a direction approximately radial to the roller ball and urges the roller ball in the direction of the fluid delivery orifice. When a user urges the device 1 against the surface of skin (not shown), a pressure is applied to the roller balls and each roller ball is urged against the means away from the fluid delivery orifice. At least a point of contact remains between the roller ball and skin (not shown). As the user moves the device 1 in one direction in the direction of the plow tips of the leading edge or the trailing edge, a torque is exerted on roller balls 16, which turn past the wicking element. Each wicking element is in fluid communication with the chamber 2 and a fluid is disposed within the chamber (not shown). Fluid penetrates the wicking elements and each wicking element deposits fluid onto the surface of the roller ball as it rotates past the portion of the wicking element with which it is in contact. As a portion of the roller ball deposits fluid on skin, the opposite portion receives more fluid from the wicking element.

As the user moves the device in one of the directions of the plow elements, hair passes through the gaps 15, as set forth supra, and is collected in the hair flow passages 14. In embodiments, a bundle of hair gathers in each hair flow passage, as shown in FIG. 6. Advantageously, the gaps 15 are smaller than the greatest distances between adjacent hair plow elements 25 (shown e.g. in FIG. 3). The motion of the device applies forces that cause the hair to be displaced and disposed into the hair flow passages 14. The smaller size of gap 15 than maximum distance between adjacent stems 25 assists in retention of hair within the hair flow passages once the hair is displaced into the passages. In embodiments, the ratio of gap 15 to distance 25 is from about 0.23 to about 0.96, in embodiments from about 0.25 to about 0.70, in embodiments from about 0.3 to about 0.6, or in embodiments from about 0.34 to about 0.51.

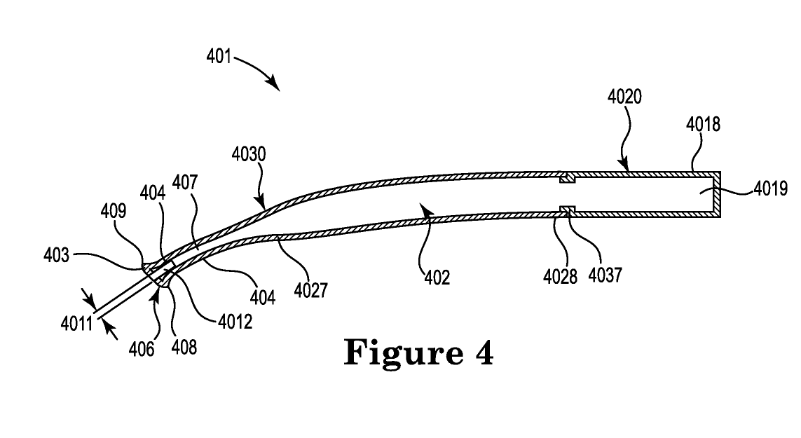

FIG. 4 shows a side-view of an embodiment of the invention, wherein each fluid delivery element 4012 comprises, consists of, or consists essentially of a wicking element 4013. A fluid supply element 4018 is hollow and defines a fluid reservoir 4019. The fluid supply element 4018 is removably attached at end 4037 of fluid reservoir 4019 to end 4028 of body 4030 by any one or more of attachment means known in the art. In some embodiments, fluid supply element 4018 is attached to end 4028 by a screw thread, in embodiments a bayonet fitting, and/or in embodiments one or more clips. In embodiments, fluid reservoir 4019 has a volume greater than or equal to chamber 402. In some embodiments, fluid supply element 4018 is supplied to the user as part of a refill, wherein the refill comprises a hollow removable container 4020, the hollow removable container defining a fluid reservoir therein and having a cap or seal therefor (not shown) attached to end 4037; in embodiments, a fluid is disposed within reservoir 4019 thereof. In some embodiments, a cap or seal is removably attached to end 4028 of body 4030: a user removes the cap or seal attached to end 4028, cap or seal attached to end 4037 of fluid supply element 4018, and attaches the fluid supply element to the input end 4028 of the body 4030 of device 401. In embodiments, the cap comprises a pressure-equalization valve.

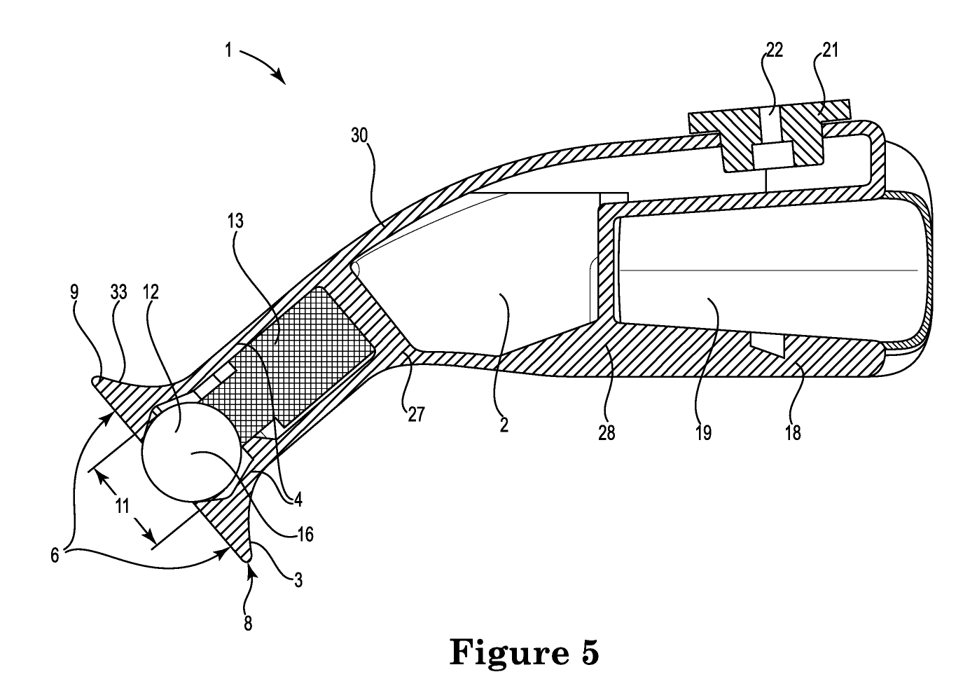

In other embodiments (not shown in FIG. 4), fluid supply element 4018 is replaced by a removable cap 21 and the chamber 2 is the fluid reservoir 19. In some embodiments, the cap 21 is affixed to end 28 of body 30 by means known in the art such as screw thread. In other embodiments, input end 28 is sealed and body 30 defines an opening in fluid communication with chamber 2. One such embodiment is shown in FIG. 5. The opening is in any convenient position in body 30 In embodiments, body 30 has a top surface 31 and a bottom surface 32 (as shown for example in FIG. 1) and the opening is in the top surface. Cap 21 is removable and disposed in the opening. In some embodiments, cap 21 comprises a first screw thread, and a second screw thread is defined by the body and disposed around the circumference of opening, wherein the cap is fixed to the device by engaging the first screw thread and the second screw thread, wherein the opening is sealed to egress of fluid from the chamber. In some embodiments, a pressure-equalization valve 22 is disposed in cap 21, as shown in FIG. 5. In other embodiments (not shown), pressure-equalization valve 22 is disposed within the body. The pressure-equalization valve is a one-way valve that allows air to enter the chamber 2 but prevents fluid disposed within the chamber from flowing or otherwise leaking therefrom.

As set forth supra, in some embodiments the fluid delivery element is a roller ball wherein a fluid wicking element 13 absorbs fluid from chamber 2. In such embodiments, the wicking element defines a path for fluid to travel from the chamber 2 to the fluid delivery element. The wicking element extends from the chamber to the roller ball and is in contact therewith. As a user presses device 1 against skin, hair or other surface, the roller ball is pushed further into the fluid delivery conduit against means 35. As the user moves the device against the skin, hair, or other surface, a point or area of the exposed surface of the roller ball is in contact therewith, and friction between the point or area causes the roller ball to rotate. As the roller ball rotates past the wicking element, fluid flows from chamber, through the wicking element, and deposits on the surface of the roller ball as the surface rotates past the wicking element and provides roller ball surface wetted with fluid. Subsequently, as user continues to move the device in one direction of the plow tips, the roller ball surface wetted with fluid rotates into fluid delivery orifice and past a portion of skin, hair, or other surface and deposits fluid thereon.

In other embodiments, the fluid delivery element consists of the wicking element. In such embodiments, the wicking element extends from the chamber to the fluid delivery orifice and at least a portion of the wicking element is either level with the fluid delivery orifice or protrudes therefrom. In such embodiments, as a user moves the device in one direction of the plow tips and urges the skin contact surfaces against skin, hair, or other surface, the wicking element qua fluid delivery element moves along the skin, hair, or other surface and fluid deposits thereon. Fluid flows from chamber into wicking element and onto the skin, hair, or other surface.

Applicants have discovered that a sintered plastic wick or silica wick is effective as the wicking element of the invention. In some embodiments, a sintered plastic wick or silica wick is used as the fluid delivery element. In other embodiments, a sintered plastic or silica wick is used in conjunction with a roller ball, as set forth supra. Advantages of sintered plastic or silica wick in conjunction with a roller ball include relative lack of dependence of amount of fluid deposited onto the skin, hair, or other surface on the speed at which the user moves the device with respect to the skin, hair, or other surface. Further, the wick acts as an intermediate reservoir of fluid between the chamber and roller ball and keeps the roller ball supplied with liquid when the device is oriented in a direction other than vertically down, as might happen for a short period of time during use. The acting as an intermediate reservoir prevents the roller ball being starved of liquid and prevents the roller ball ceasing to dispense.

In embodiments, the sintered plastic of the sintered plastic wick is made of sintered polyethylene. Such materials are commercially available, for example as Vyon® sintered porous plastic materials, available from Porvair Filtration Group Inc. of Ashland, Va. The pore size can be tailored for the desire flow rate for a particular fluid. Further, in some embodiments of the device including one or more roller balls, each roller ball and associated fluid delivery orifice together define a fluid delivery gap therebetween—in embodiments the gap is zero or substantially zero when the device is not in use. When the user applies a roller ball to a portion of skin and/or hair, as set forth supra, the pressure applied by the user to the device urges the roller ball away from the fluid delivery orifice against the means for urging roller ball into its associated fluid orifice, whereby a fluid delivery gap between the roller ball and the orifice and defined therebetween is opened. In embodiments where the means is a spring, the greater the force with which the user applies the device to the portion of skin and/or hair, the greater the fluid delivery gap. In such embodiments, the user can control the flowrate of the fluid with the force.

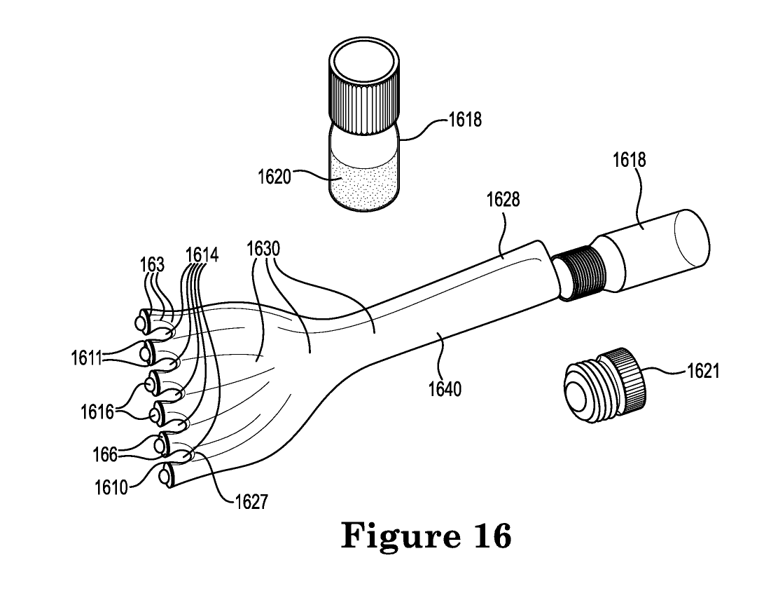

FIG. 16 shows a fluid application assemblage comprising a device embodiment of the invention for application of a fluid to a skin surface with hair covering, wherein the device comprises a hollow manifold body 1630 surrounding and defining a chamber therein and comprising an input end 1628 and an output end 1627, and a plurality of hair plow elements 163 attached to and extending away from the output end of the manifold body and arranged in a generally linear array with a spacing interval between adjacent hair plow elements, wherein each hair plow element comprises a skin contact surface 166 and a hollow stem extending from the skin contact surface toward the manifold body, the stem connecting the element to the output end, each hollow stem surrounding and defining a fluid delivery conduit within the stem, the conduit being in fluid communication with the chamber, the skin contact surface of each hair plow element comprising a leading edge comprising a plow tip 1610, the skin contact surface defining a fluid delivery orifice 1611 with a roller ball 1616 element positioned in the orifice and disposed within the conduit, and adjacent stems defining a hair flow passage 1614 therebetween. Body 1630 narrows towards input end 1628 forming and comprising a hollow handle 1640. Input end 1628 is adapted to receive a fluid supply element 1618, endcap 1621, or both the fluid supply element and the endcap.

The assemblage comprises, consists of, or consists essentially of the device. In embodiments, the assemblage comprises the device and a further item selected from an endcap, a refill, or both an endcap and a refill, wherein the refill comprises, consists of, or consists essentially of a hollow removable container, a container-cap removably attached to one end of the removable container, and a fluid disposed within the hollow container. In embodiments, the assemblage comprises, consists of, or consists essentially of the device and an additional item selected from fluid supply element 1618, endcap 1621, container-cap, or any combination thereof. In some such embodiments of the assemblage, the fluid supply element is removably attached to input end 1628. In the embodiments shown in FIG. 16, the fluid supply element comprises, consists of, or consists essentially of a removable container 1620. In embodiments, the fluid supply element comprises, consists of, or consists essentially of hollow removable container 1620 defining a fluid reservoir therein and a fluid for application to a skin surface bearing hair disposed in said container within said fluid reservoir. In some such embodiments, the fluid supply element 1618 is supplied as part of a refill, the refill comprising removable container 1620, a fluid disposed within the container, and a removable container-cap removably attached to one end of the container. In embodiments, the assemblage includes endcap 1621 adapted for attachment to the device at end 1628. In some such embodiments of the assemblage, the endcap 1621 is removably attached to the device at end 1628. In some such embodiments, the endcap 1621 is removed from the device before attaching the fluid supply element 1618. The removable container 1620 is adapted to attach to input end 1628 by attachment means such as a screw thread and adapted or further adapted to receive the container-cap.

In embodiments, the assemblage comprises the device and the fluid supply element 1618 removably attached to end 1628 by a screw thread. In some such embodiments, the fluid supply element consists of or consists essentially of the removable container 1620, wherein the removable container is hollow thereby defining a fluid reservoir. In such embodiments of the assemblage, the fluid reservoir is in fluid communication with the chamber, the fluid delivery conduits, and the roller balls 1616. In embodiments, the endcap comprises a pressure-equalization valve.

In embodiments, the assemblage comprises the device and the endcap 1621 removably attached to input end 1628, and a fluid is disposed within the chamber of the device.

The device can be used by a user on a subject. In some embodiments, the user is the subject; that is to say that the user applies fluid to his or her own skin. In other embodiments, the user is separate from the subject. In such embodiments, the subject is a human or non-human animal (herein “animal”).

Fluids

Almost any fluid adapted and/or designed for application to the scalp or scalp and hair can be applied by the device and methods of the invention: In embodiments, the fluid is a fluid formulated for topical application to the skin and/or hair, the fluid selected from a nutraceutical preparation, a cosmetic preparation, a medicinal preparation, a parasite repellant, or a parasiticide. Examples include Nutriol® Hair Fitness Treatment, sold by NU SKIN® Enterprises of Provo, Utah; NU SKIN® Conductive Gel, sold by NU SKIN® ® Enterprises of Provo, Utah; fluids formulated and/or adapted to mitigate or reverse hair loss; medicinal fluids formulated and/or adapted for treatment of psoriasis, dandruff, and eczema; fluids formulated and/or adapted to combat, repel, and/or kill parasites such as flea and/or tick repellant and lice treatment agents; fluids formulated and/or adapted for relief of itch; fluids formulated and/or adapted for nourishment of the scalp and/or hair; fluids formulated and/or adapted for amelioration of symptoms and/or damage from sunburn; and fluids formulated and/or adapted to nourish the skin including fluids designed to hydrate, moisturize, and/or provide antioxidants and/or vitamins to the skin. In embodiments there is provided an apparatus comprising any of the embodiments of the device described herein and a fluid selected from Nutriol® Hair Fitness Treatment, sold by NU SKIN® ® Enterprises of Provo, Utah; NU SKIN® Conductive Gel, sold by NU SKIN® ® Enterprises of Provo, Utah; hair loss treatment, psoriasis treatment, dandruff treatment, eczema treatment, parasite repellant, parasiticide, itch-relief medicament, nutraceutical, sunburn treatment, moisturizer, analgesic, or any combination thereof.

In embodiments, the dynamic viscosity of the fluid is about 0.1 cP to about 10 cP between 15 and 40° C., in embodiments about 0.3 cP to about 10 cP.

In embodiments, the fluid is thixotropic.

### Second Embodiments

In some embodiments, device 1 further comprises a transcutaneous electrical stimulator (TES). The transcutaneous electrical stimulator comprises a plurality of contact electrodes in an array and a source of electrical current. Transcutaneous electrical stimulators apply a low-intensity electrical current to the skin when used in combination with a conductive fluid applied between an contact electrode and the skin. A transcutaneous electrical stimulator may be galvanic, that is capable of delivering a direct current, or may deliver an alternating current similarly to a transcutaneous electrical nerve stimulation device. Passing an electric current through an electrically conductive fluid disposed between an contact electrode and the skin may directly effect changes in the fluid, skin, or both fluid and skin. In some embodiments, depending on the chemical composition of the electrically conductive fluid, passing a current through the fluid may effect a chemical change in the fluid, which in turn effects a beneficial or therapeutic chemical and/or physical change in the skin. Although the device of the present invention is configured to preferentially apply a fluid to a skin surface that bears hair rather than said hair on the skin surface, changes effected to fluid by transcutaneous electrical stimulation can also be beneficial for hair. In the present invention, in some embodiments one or more skin contact surfaces each comprises a contact electrode conformed thereto. In some such embodiments, every skin contact surface of the device comprises, consists of, or consists essentially of a contact electrode. During application of a fluid to skin, a film or layer of fluid collects between contact electrodes and skin and/or hair. If the fluid is electrically conductive, application of a voltage to the contact electrodes causes a current to flow through the film or layer of fluid disposed between an contact electrode and the skin and/or hair. Non-limiting examples of changes effected to a fluid disposed between an contact electrode and hair and/or skin by passing of a current therethrough include polymerization and/or gelling of fluid and/or components of the fluid, ionization of the fluid and/or components thereof, oxidation and/or reduction of the fluid and/or components thereof, electro-osmosis, and the like. A transcutaneous electrical stimulator may be iontophoretic when coupled with a suitable conductive fluid including one or more ionic compounds with therapeutic or medicinal effects. Exemplary transcutaneous electrical stimulators comprise, consist essentially of or consist of devices that comport or substantially comport with the U.S. Food and Drug Agency's Regulation Number 882.5890. Exemplary transcutaneous electrical stimulators include but are not limited to ageLOC® Body Spa, sold by NU SKIN® ® Enterprises of Provo, Utah. In embodiments, the transcutaneous electrical stimulator delivers a low-intensity, low-frequency electrical current to the skin when used in combination with a conductive fluid applied between the source of the current and a selected treatable skin portion of a human subject. Transcutaneous electrical stimulators are known in the industry. Examples of transcutaneous electrical stimulator devices include but are not limited to those described in U.S. Pat. Nos. 6,119,038; 6,766,199; 7,305,269, the entirety of which are incorporated herein by reference; the ageLOC® Body Spa, sold by NU SKIN® Enterprises of Provo, Utah; and the NU SKIN® Facial Spa, also sold by NU SKIN® Enterprises. In some embodiments, the source of current includes one or more controllers that enable the user to vary or modulate one or more of a pulse width, a frequency, or an intensity of the electrical current delivered to the skin of a subject.

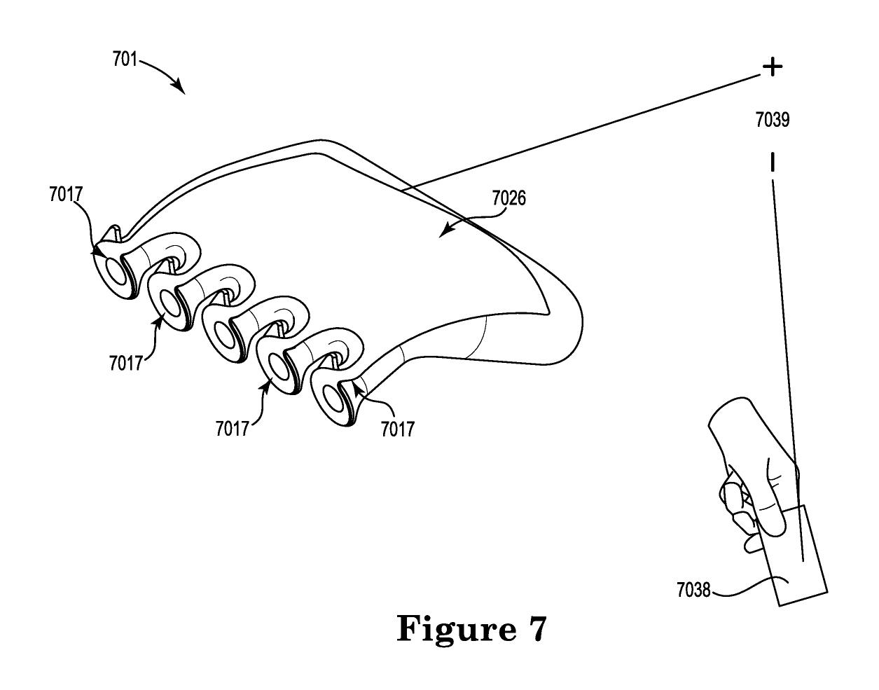

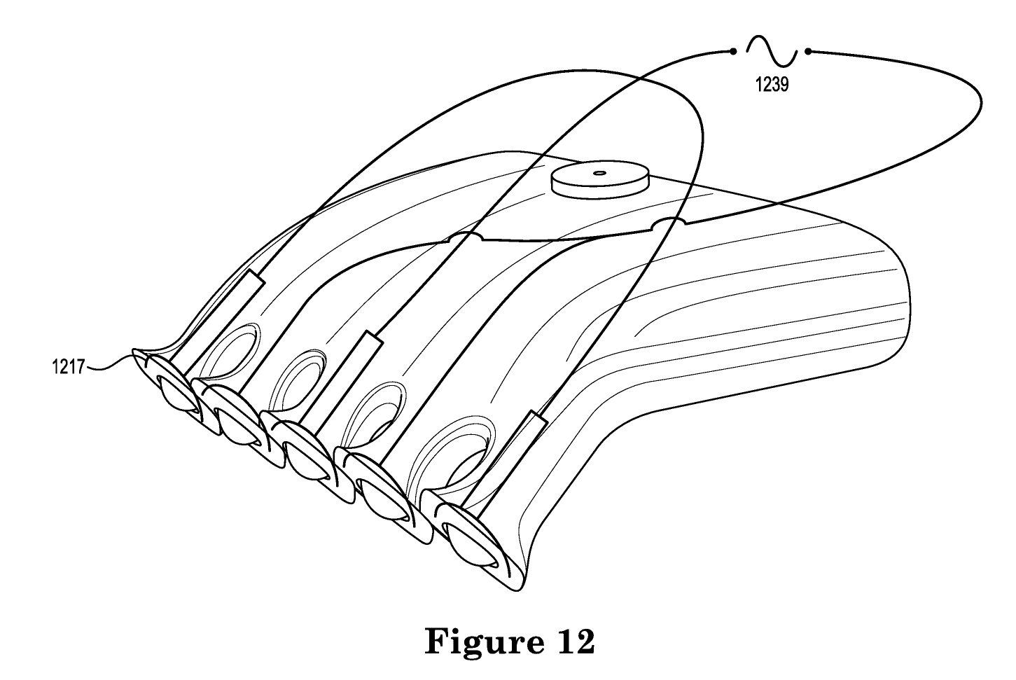

In embodiments, device for application of a fluid to a skin surface with hair covering comprises two or more contact electrodes 7017 (see FIG. 7.). In some such embodiments, a part of every skin contact surface 6 comprises a contact electrode 17, as shown in FIGS. 7 and 12, which depict two exemplary embodiments of the invention. In other embodiments, the entire skin contact surface of each hair plow element comprises a contact electrode. In such embodiments, the device therefore comprises a plurality of contact electrodes disposed on the plurality of hair plow elements.

The power source is configured to apply a voltage across at least two terminals and supply a current to a circuit. The power source is not particularly limited and may be a source of direct current or a source of alternating current. Current may be fixed or variable. Variable current may be monophasic or biphasic (two phase), alternating or half-wave rectified. The source comprises two terminals, one terminal at an electrical potential (versus the earth) V₁ and one terminal at an electrical potential V₂ (versus earth). The power source may be any electrical supply as is known in the art, and may include controller circuitry (for selecting and controlling voltage, current, polarity or waveform and/or one or more control switches or other input devices. The power source may be embodied in, attached to, or otherwise incorporated in device; or the power source may be a separate unit electrically connected to the device by one or more wires.

Embodiments including TES with a common electrode.

In some embodiments wherein device 701 comprises contact electrodes 7017, each contact electrode 7017 is electrically connected to a single common electrode 7026 that is in contact with a first terminal of the source 7039—one such embodiment is represented schematically in FIG. 7. In such embodiments, the contact electrodes are collectively equal to each other or substantially equal to each other with respect to their electrical potential relative to earth. In use, a user holds device 701, applies the device to an area of the skin and/or hair of a subject, and the skin contact surfaces and the contact electrodes that they comprise collectively contact the skin and/or hair of the subject. In such embodiments, the subject (whether the user or someone else) holds and/or touches second common electrode 7038 that is in contact electrode with a second terminal of the source. In embodiments, the second common electrode is unattached to or detached from device 701 although forming part of an apparatus—in such embodiments, the apparatus comprises, consists of, or consists essentially of device 701 and second common electrode 7038. In embodiments wherein second common electrode 7038 is not attached to device 701, the subject is in in electrical contact with second common electrode 7038, for example subject holds second common electrode 7038. In other embodiments wherein the user is also the subject, in some embodiments the second common electrode is attached to device 701 on the body 7030, the fluid reservoir 7019, or any convenient part of device 701 at a location where the user can conveniently hold the device and simultaneously be in contact electrode with second common electrode 7038 with one hand.

In embodiments wherein device comprises a second common electrode, user grips device in his or her own hand and is in contact electrode with second common electrode 7038 and completes an electrical circuit. The electrical circuit comprises an electrical source 7039. The electrical source applies an electromotive force to the circuit. When an electromotive force of sufficient voltage is applied to the circuit and device 701 is applied to an area of skin, current flows through each electrode 7017, through the area of skin in contact with the electrode or through a film of fluid disposed between skin contact surface and an area of the skin, through the body and arm of the subject, into second common electrode 7038, and returns to source 7039; or the current flows in the opposite direction, depending on the source. In some embodiments, the source applies a consistent or substantially consistent electrical potential to terminals and the source is a source of direct current. In other embodiments, the source varies the electrical potentials V₁ and V₂.

In embodiments, common electrode 7026 and contact electrodes 7017 are attached to body 7030 of device 701 and conformed in shape thereto—such an embodiment is shown schematically in FIG. 7.

Embodiments including a TES without common electrode.

In embodiments, contact electrodes in a plurality of contact electrodes do not have the same electrical potential as each other. In such embodiments, a second common electrode is not required. In some such embodiments, the electrodes in the plurality of electrodes alternate in connection to the first terminal and second terminal of the source 1239. In such embodiments, alternating contact electrodes in the array always have the same electrical polarity as each other and adjacent pairs of contact electrodes have opposing polarity or zero polarity. In embodiments wherein the source 1239 is a source of an alternating current, the potential of contact electrodes changes with time. When the source 1239 is a source of a single-phase sinusoidally varying alternating current and the contact electrodes in the plurality of electrodes alternate in connection to the first terminal and second terminal of the source, the potential of a pair of adjacent contact electrodes changes with time and is opposite at 90° and 270° phase, but equal and zero with respect to earth at 0°, 180°, and 360° phase.

In such embodiments, source 1239 is a source of direct current or a source of alternating current. One such embodiment wherein source is a source of alternating current is illustrated schematically in FIG. 12. When source 1239 is a source of direct current, each electrode of a pair of adjacent electrodes has a different electrical potential from the other. When source is a source of alternating current, each electrode of a pair of adjacent electrodes has a different electrical potential from the other at least part of the time. When device 121 comprising a transcutaneous electrical stimulator is applied to an area of skin bearing hair, the potential difference between pairs of adjacent electrodes causes a current to flow therebetween. When the source 1239 generates an alternating current in the circuit, the electrical polarity of each contact periodically changes. If the alternating current is a single phase, then the polarity of each contact electrode changes in phase with all of the others.

In some embodiments, the transcutaneous electrical stimulator is designed and adapted to generate a low-frequency, low-intensity, sustained current that is passed in both anodal and cathodal directions relative to a selected treatable skin portion contacted by the transcutaneous electrical stimulator during conductively engaged contact thereof. In some embodiments, the average current intensity delivered by the transcutaneous electrical stimulator is about 1 mA or less at 2 kOhm, for example about 0.1 mA to 1 mA, or about 0.2 mA to 1 mA, or about 0.3 mA to 1 mA, or about 0.4 mA to 1 mA, or about 0.1 mA to 0.9 mA, or about 0.1 mA to 0.8 mA, or about 0.1 mA to 0.7 mA, or about 0.1 mA to 0.6 mA, or about 0.1 mA to 0.5 mA, or about 0.1 mA to 0.4 mA, or about 0.2 mA to 0.8 mA, or about 0.2 mA to 0.6 mA at 2 kOhm. In some embodiments the waveform of the current and/or voltage generated by the source of electrical current is pulsed monophasic. In some embodiments the waveform of the current and/or voltage generated by the source of electrical current is modulated monophasic, which means symmetrically separated into an anodal phase and a cathodal phase. In such embodiments the total current per phase is about 80 mA-s to 200 mA-s for each phase, such as about 100 mA-s to 150 mA-s for each phase; wherein the total current time per phase correlates to about 80 mC to 200 mC per phase, such as about 100 mC to 150 mC for each phase. In such embodiments, the total mC is the sum of the mC for each of the two phases. In some embodiments the maximum output voltage of the transcutaneous electrical stimulator is less than about 200 mV at 500 ohm during conductively engaged contact, such as about 100 mV to 200 mV, or 120 mV to 200 mV, or about 100 mV to 150 mV, or about 150 mV to 200 mV at 500 ohm during conductively engaged contact. In some embodiments, the transcutaneous electrical stimulator current output is between 250 μA and 400 μA at 500 ohm during conductively engaged contact; in some such embodiments, the transcutaneous electrical stimulator includes one or more controllers for the user to adjust or select an output from 0 to about 400 μA.

In embodiments, the source 1239 of current further comprises a switch configured so that current is transferred to the skin from the contact electrodes only when the switch is closed. The circuit is completed with contact to the skin, whereas the open circuit has no current. In embodiments the transcutaneous electrical stimulator and/or source of electrical current further includes an automatic overload trip and/or an automatic no-load trip safety feature. Such safety features are well known in the industry, as will be understood by those of skill.

The contact electrodes are formed from or include a metal or metal mixture or other electrically conductive material suitable to conduct electricity, that is, suitable to deliver an electrical current from the source of electrical current to the skin when a conductive fluid is disposed between the contact electrodes and the selected treatable skin surface. In embodiments, the contact electrodes comprise any material with the proviso that the impedance of the material is negligible compared with the skin. In embodiments, the contact electrodes have an impedance from about zero to about 500 ohms, in embodiments from about 0.1 ohms to about 100 ohms, in embodiments from about 0.1 ohms to about 10 ohms. Suitable electrically conductive materials include stainless steel, copper and copper alloys, chrome-plated polymers, gold-plated polymers, nickel-plated metals, silver-plated metals, graphite, or conductive polymers.

In the second embodiments, at least a part of each of one or more skin contact surfaces of the device comprises, consists of, or consists essentially of a contact electrode. In an aspect generally in the direction of the stems (such as the aspect shown schematically in FIG. 2 showing an embodiment without transcutaneous electrical stimulator), the contact electrodes are only limited by the shape and size of the stems, and the shape and size of the fluid dispersion orifices defined by the skin contact surfaces: each contact electrodes is of a shape and size such that, in embodiments, it is bounded by the total area of the skin contact surface of which it forms a part and does not extend over the edge of the area in the aspect viewed generally in the direction of the stems. No contact electrode covers a fluid dispersion orifice. In the aspect, in embodiments the contact electrodes are generally circular, elliptical, parabolic, hyperbolic, U-shaped, or some other regular or irregular shape. In embodiments, the surface area of each contact electrode is substantially flat or slightly convex

In some embodiments, the transcutaneous electrical stimulator is iontophoretic when coupled with a suitable conductive fluid further including one or more ionic compounds with therapeutic or medicinal effects.

### Method Embodiments

In embodiments, there is provided a method of application of a fluid to skin bearing hair, the method comprising, consisting of, or consisting essentially of: applying the skin contact surfaces of any of the embodiments of the device of the invention described herein to a first area of skin and/or hair of a human or animal subject, moving the device in one of either of the directions of the plow tips for a first period of time while maintaining contact between the skin contact surfaces and a second area of skin and/or hair, and removing the skin contact surfaces from the skin; wherein at least some of the fluid is deposited as a layer on some or all of the second area of skin and/or hair. In embodiments, the first period of time is from about one second to about ten seconds. In embodiments, the method further comprises repeating the applying, moving, and removing in the sequence of applying, moving, and removing in any number of repetitions over an additional treatment time. The total treatment time is the time from the applying the skin contact surfaces to the first area of skin and/or hair to the end of the additional treatment time. In embodiments, the total treatment time is about 1 minute to one hour in duration, or about 1 minute to 45 minutes, or about 1 minute to 30 minutes, or about 1 minute to 15 minutes, or about 1 minute to 10 minutes, or about 1 minute to 8 minutes, or about 1 minute to 5 minutes, or about 2 minutes to 20 minutes, or about 5 minutes to 20 minutes, or about 5 minutes to 15 minutes, or about 5 minutes to 12 minutes, or about 5 minutes to 10 minutes, or about 7 minutes to 15 minutes, or about 7 minutes to 12 minutes in duration. In embodiments, one or more other steps intervenes between applying and moving. In embodiments, the time between repeats, that is the time between moving and applying is from about one second to about one minute.

The skin contact surfaces are in contact with a third area of skin and/or hair, wherein the third area of skin is the total area of the skin contacted by the skin contact surfaces during the total treatment time.

In embodiments, the method further comprises filling or partially filling the chamber of any of the embodiments of the device of the invention described herein with any of the fluids described herein. In some embodiments, the filling of the chamber is effected by attachment of a fluid supply element to the input end of the device, wherein the fluid supply element defines a fluid reservoir having fluid disposed therein. In other embodiments, the filling of the chamber is effected by pouring or pumping fluid from a source of fluid through an opening defined by the body into the chamber. In embodiments, the source of fluid comprises fluid and a containment therefor. In embodiments, at least some of the first area of skin, the second area of skin, or both the first and second areas of skin bears hair. In embodiments, more fluid is applied to the second area of skin than to hair.

In embodiments, the total surface area of skin and/or hair engaged by contact with the skin contact surfaces during the total treatment time is about 1 m² to 1 cm², or about 0.5 m² to 1 cm², or about 0.4 m² to 1 cm², or about 0.3 m² to 1 cm², or about 0.2 m² to 1 cm², or about 0.1 m² (1000 cm²) to 1 cm², or about 900 cm² to 1 cm², or about 800 cm² to 1 cm², or about 700 cm² to 1 cm², or about 600 cm² to 1 cm², or about 500 cm² to 1 cm², or about 400 cm² to 1 cm², or about 300 cm² to 1 cm², or about 200 cm² to 1 cm², or about 100 cm² to 1 cm², or about 50 cm² to 1 cm², or about 40 cm² to 1 cm², or about 30 cm² to 1 cm², or about 20 cm² to 1 cm², or about 10 cm² to 1 cm², or about 9 cm² to 1 cm², or about 8 cm² to 1 cm², or about 7 cm² to 1 cm², or about 6 cm² to 1 cm², or about 5 cm² to 1 cm², or about 4 cm² to 1 cm², or about 3 cm² to 1 cm², or about 2 cm² to 1 cm², or about 1000 cm² to 2 cm², or about 1000 cm² to 3 cm², or about 1000 cm² to 4 cm², or about 1000 cm² to 5 cm², or about 1000 cm² to 6 cm², or about 1000 cm² to 7 cm², or about 1000 cm² to 8 cm², or about 1000 cm² to 9 cm², or about 1000 cm² to 10 cm², or about 1000 cm² to 20 cm², or about 1000 cm² to 30 cm², or about 1000 cm² to 40 cm², or about 1000 cm² to 50 cm², or about 1000 cm² to 60 cm², or about 1000 cm² to 70 cm², or about 1000 cm² to 2 cm², or about 1000 cm² to 80 cm², or about 1000 cm² to 90 cm², or about 1000 cm² to 100 cm², or about 1000 cm² to 200 cm², or about 1000 cm² to 300 cm², or about 1000 cm² to 400 cm², or about 1000 cm² to 500 cm², or about 1000 cm² to 600 cm², or about 1000 cm² to 700 cm², or about 1000 cm² to 800 cm², or about 1000 cm² to 900 cm². or about 500 cm² to 10 cm², or about 500 cm² to 100 cm², or about 500 cm² to 50 cm².

### Further Method Embodiments

In embodiments, there is provided a method of application of a fluid to skin bearing hair, the method comprising, consisting of, or consisting essentially of: applying the skin contact surfaces of the device of any of the second embodiments of the invention to a first area of skin and/or hair of a human or animal subject; moving the device in one of either of the directions of the plow tips for a first period of time while maintaining contact between the skin contact surfaces and a second area of skin and/or hair, maintaining conductively engaged contact between the contact electrodes and at least a portion of the second area of skin and/or hair; and removing the contact electrodes from the skin; wherein at least some of the fluid is deposited as a layer on some or all of the second area of skin and/or hair. In embodiments, the first period of time is from about one second to about ten seconds. In embodiments, the method further comprises repeating the sequence of steps comprising applying, moving, and removing in any number of repetitions over an additional treatment time, wherein any individual repetition of the sequence optionally comprises a conductively engaged contact between the contact electrodes and a portion of the skin. The total treatment time is the time from the applying the skin contact surface to the first area of skin to the end of the additional treatment time. In embodiments, the total treatment time is about 1 minute to 20 minutes in duration, or about 1 minute to 18 minutes, or about 1 minute to 15 minutes, or about 1 minute to 12 minutes, or about 1 minute to 10 minutes, or about 1 minute to 8 minutes, or about 1 minute to 5 minutes, or about 2 minutes to 20 minutes, or about 5 minutes to 20 minutes, or about 5 minutes to 15 minutes, or about 5 minutes to 12 minutes, or about 5 minutes to 10 minutes, or about 7 minutes to 15 minutes, or about 7 minutes to 12 minutes in duration. In embodiments, one or more other steps intervenes between applying and moving. In embodiments, the time between repeats, that is the time between moving and applying is from about one second to about one minute. The skin contact surfaces are in contact with a third area of skin, wherein the third area of skin is the total area of the skin and/or hair contacted by the skin contact surfaces during the total treatment time.

In embodiments, the method further comprises filling or partially filling the chamber of any of the embodiments of the device of the invention described herein with any of the fluids described herein. In some embodiments, the filling of the chamber is effected by attachment of a fluid supply element to the input end of the device, wherein the fluid supply element defines a fluid reservoir having fluid disposed therein. In other embodiments, the filling of the chamber is effected by pouring or pumping fluid from a source of fluid through an opening defined by the body into the chamber. In embodiments, the source of fluid comprises fluid and a containment therefor. In embodiments, at least some of the first area of skin, the second area of skin, or both the first and second areas of skin bears hair. In embodiments, more fluid is applied to the second area of skin than to hair.

The conductively engaged contact comprises, consists essentially of, or consists of contact between the conductive head of the transcutaneous electrical stimulator as current is delivered from the head portion to an area of the selected treatable skin portion. The transcutaneous electrical stimulator is suitably turned “on”—caused to conduct electricity to the head portion—before, after, or contemporaneously with the contacting to result in the conductively engaged contact.

In embodiments, the total period of conductively engaged contact within the total treatment time is about 2 minutes to 20 minutes in duration, or about 2 minutes to 18 minutes, or about 2 minutes to 15 minutes, or about 2 minutes to 12 minutes, or about 2 minutes to 10 minutes, or about 2 minutes to 8 minutes, or about 2 minutes to 5 minutes, or about 3 minutes to 20 minutes, or about 5 minutes to 20 minutes, or about 5 minutes to 15 minutes, or about 5 minutes to 12 minutes, or about 5 minutes to 10 minutes, or about 7 minutes to 15 minutes, or about 7 minutes to 12 minutes in duration, further wherein the conductively engaged contact is distributed by the user over the selected treatable skin portion. Temporally, in some embodiments the conductively engaged contact is distributed substantially evenly over the entirety of the third area. The user causes the distribution by moving the contact electrodes over at least a portion of the third area of skin and/or hair.

In some embodiments the conductively engaged contact is distributed substantially evenly over the entirety of the third area in terms of time of conductive engagement per unit of skin surface area engaged. In other embodiments, the user may choose to apply the conductively engaged contact unevenly in terms of time of engaged contact per unit of skin surface area engaged. The user causes the selected distribution of engaged contact by moving the contact electrodes around the selected treatable skin portion while maintaining conductively engaged contact with the skin surface.

In some embodiments, the total surface area of skin conductively engaged by contact during the total treatment time is about 1 m² to 1 cm², or about 0.5 m² to 1 cm², or about 0.4 m² to 1 cm², or about 0.3 m² to 1 cm², or about 0.2 m² to 1 cm², or about 0.1 m² (1000 cm²) to 1 cm², or about 900 cm² to 1 cm², or about 800 cm² to 1 cm², or about 700 cm² to 1 cm², or about 600 cm² to 1 cm², or about 500 cm² to 1 cm², or about 400 cm² to 1 cm², or about 300 cm² to 1 cm², or about 200 cm² to 1 cm², or about 100 cm² to 1 cm², or about 50 cm² to 1 cm², or about 40 cm² to 1 cm², or about 30 cm² to 1 cm², or about 20 cm² to 1 cm², or about 10 cm² to 1 cm², or about 9 cm² to 1 cm², or about 8 cm² to 1 cm², or about 7 cm² to 1 cm², or about 6 cm² to 1 cm², or about 5 cm² to 1 cm², or about 4 cm² to 1 cm², or about 3 cm² to 1 cm², or about 2 cm² to 1 cm², or about 1000 cm² to 2 cm², or about 1000 cm² to 3 cm², or about 1000 cm² to 4 cm², or about 1000 cm² to 5 cm², or about 1000 cm² to 6 cm², or about 1000 cm² to 7 cm², or about 1000 cm² to 8 cm², or about 1000 cm² to 9 cm², or about 1000 cm² to 10 cm², or about 1000 cm² to 20 cm², or about 1000 cm² to 30 cm², or about 1000 cm² to 40 cm², or about 1000 cm² to 50 cm², or about 1000 cm² to 60 cm², or about 1000 cm² to 70 cm², or about 1000 cm² to 2 cm², or about 1000 cm² to 80 cm², or about 1000 cm² to 90 cm², or about 1000 cm² to 100 cm², or about 1000 cm² to 200 cm², or about 1000 cm² to 300 cm², or about 1000 cm² to 400 cm², or about 1000 cm² to 500 cm², or about 1000 cm² to 600 cm², or about 1000 cm² to 700 cm², or about 1000 cm² to 800 cm², or about 1000 cm² to 900 cm². or about 500 cm² to 10 cm², or about 500 cm² to 100 cm², or about 500 cm² to 50 cm².

In some embodiments, the total surface area of skin conductively engaged per unit of time within the total treatment time is about 1 cm²/minute to 400 cm²/minute, or about 1 cm²/minute to 350 cm²/minute, or about 1 cm²/minute to 300 cm²/minute, or about 1 cm²/minute to 250 cm²/minute, or about 1 cm²/minute to 200 cm²/minute, or about 1 cm²/minute to 150 cm²/minute, or about 1 cm²/minute to 100 cm²/minute, or about 1 cm²/minute to 50 cm²/minute, or about 1 cm²/minute to 40 cm²/minute, or about 1 cm²/minute to 30 cm²/minute, or about 1 cm²/minute to 20 cm²/minute, or about 1 cm²/minute to 10 cm²/minute, or about 1 cm²/minute to 9 cm²/minute, or about 1 cm²/minute to 8 cm²/minute, or about 1 cm²/minute to 7 cm²/minute, or about 1 cm²/minute to 6 cm²/minute, or about 1 cm²/minute to 5 cm²/minute, or about 1 cm²/minute to 4 cm²/minute, or about 1 cm²/minute to 3 cm²/minute, or about 1 cm²/minute to 2 cm²/minute, or about 2 cm²/minute to 400 cm²/minute, or about 3 cm²/minute to 400 cm²/minute, or about 4 cm²/minute to 400 cm²/minute, or about 5 cm²/minute to 400 cm²/minute, or about 6 cm²/minute to 400 cm²/minute, or about 7 cm²/minute to 400 cm²/minute, or about 8 cm²/minute to 400 cm²/minute, or about 9 cm²/minute to 400 cm²/minute, or about 10 cm²/minute to 400 cm²/minute, or about 20 cm²/minute to 400 cm²/minute, or about 30 cm²/minute to 400 cm²/minute, or about 40 cm²/minute to 400 cm²/minute, or about 50 cm²/minute to 400 cm²/minute, or about 60 cm²/minute to 400 cm²/minute, or about 70 cm²/minute to 400 cm²/minute, or about 80 cm²/minute to 400 cm²/minute, or about 90 cm²/minute to 400 cm²/minute, or about 100 cm²/minute to 400 cm²/minute, or about 150 cm²/minute to 400 cm²/minute, or about 200 cm²/minute to 400 cm²/minute, or about 250 cm²/minute to 400 cm²/minute, or about 300 cm²/minute to 400 cm²/minute, or about 350 cm²/minute to 400 cm²/minute, or about 5 cm²/minute to 200 cm²/minute, or about 5 cm²/minute to 100 cm²/minute, or about 5 cm²/minute to 50 cm²/minute, or about 10 cm²/minute to 200 cm²/minute, or about 10 cm²/minute to 100 cm²/minute, or about 20 cm²/minute to 200 cm²/minute, or about 50 cm² to 200 cm²/minute, or about 100 cm² to 200 cm²/minute.

The treatment comprises the total number of applying, moving, and removing steps during the total treatment time. In embodiments, a course of treatment comprises one or more treatments. In embodiments, the treatments described are repeated. In embodiments, the treatments described are repeated once, in embodiments twice, in embodiments three times, or in embodiments four times to provide a course of treatment. In embodiments, the course of treatment is carried out at intervals over about 24 hours, in embodiments, about one week, in embodiments, about one month, in embodiments about three months, or in embodiments about one year. Treatments with no time duration are also contemplated.

The invention illustratively disclosed herein can be suitably practiced in the absence of any element which is not specifically disclosed herein. While the invention is susceptible to various modifications and alternative forms, specifics thereof have been shown by way of examples, and are described in detail. It should be understood, however, that the invention is not limited to the particular embodiments described. On the contrary, the intention is to cover modifications, equivalents, and alternatives falling within the spirit and scope of the invention. In various embodiments, the invention suitably comprises, consists essentially of, or consists of the elements described herein and claimed according to the claims.

Additionally each and every embodiment of the invention, as described here, is intended to be used either alone or in combination with any other embodiment described herein as well as modifications, equivalents, and alternatives thereof falling within the spirit and scope of the invention. The various embodiments described above are provided by way of illustration only and should not be construed to limit the claims attached hereto. It will be recognized that various modifications and changes may be made without following the example embodiments and applications illustrated and described herein, and without departing from the true spirit and scope of the claims.

What we claim is:

1. A device for application of a fluid to a skin surface with hair covering, the device comprising:
a hollow manifold body surrounding and defining a chamber therein and comprising an input end and an output end; and
a plurality of hair plow elements attached to and extending away from the output end of the manifold body and arranged in a generally linear array with a spacing interval between adjacent hair plow elements,
each hair plow element comprising a skin contact surface and a hollow stem extending from the skin contact surface toward the manifold body, the stem connecting the element to the output end,
each hollow stem surrounding and defining a fluid delivery conduit within the stem, the conduit being in fluid communication with the chamber,
the skin contact surface comprising a leading edge comprising a plow tip,
the skin contact surface defining a fluid delivery orifice with a fluid delivery element positioned in the orifice and disposed within the conduit, and
adjacent stems defining a hair flow passage therebetween.

2. The device of claim 1, wherein each hair flow passage is arcuate in an aspect perpendicular to the adjacent stems and to the skin contact surfaces, and in the aspect the distance between adjacent stems is minimum proximal to the skin contact surfaces.

3. The device of claim 1, wherein the shape of the skin contact surface is approximately a lenticular shape, reuleaux triangular, a rounded rhombus, or a pear shape.

4. The device of claim 1, wherein the plow tip is rounded off.

5. The device of claim 1, wherein the skin contact surface comprises a trailing edge and tapers outward from the leading edge around the delivery orifice and then inward toward the trailing edge.

6. The device of claim 5, wherein the skin contact surface is approximately lenticular in shape comprising a major axis and a minor axis perpendicular to each other.

7. The device of claim 6, wherein the ratio of width of the skin contact surface along the minor axis to the length of the skin contact surface along the major axis is from 6:18 to 10:12.

8. The device of claim 7, wherein the width of the skin contact surface along the minor axis is about 8.6 mm and the length of the skin contact surface along the major axis is about 12.1 mm.

9. The device of claim 8, wherein the spacing interval between adjacent hair plow elements is about 10 mm.

10. The device of claim 1, wherein the fluid delivery element is selected from: (a) a wicking element in fluid communication with the chamber and extending to the delivery orifice; or (b) a roller ball captured in the delivery orifice and in fluid communication with the chamber.

11. The device of claim 10, wherein the fluid delivery element is the roller ball captured in the delivery orifice, and wherein each plow element further comprises a wicking element located within the fluid delivery conduit and extends to and is in fluid contact with a portion of the surface of the roller ball.

12. The device of claim 1, wherein the fluid delivery element is effective for delivering a fluid with a water-like viscosity and the fluid is a cosmetic and/or a nutraceutical preparation for the skin.

13. The device of claim 1 further comprising a source of electrical current and a plurality of contact electrodes in an array, wherein each skin contact surface of a hair plow element comprises an contact electrode in electrical contact with the source.

14. The device of claim 13, wherein alternating electrodes in the array have the same electrical polarity and adjacent pairs of contact electrodes have opposing or zero polarity.

15. The device of claim 1, further comprising a hollow fluid supply element attached to the input end of the body, the hollow fluid supply element defining therein a fluid reservoir in fluid communication with the chamber, wherein the fluid supply element is selected from a hose, a fixed container, or a removable container.

16. The device of claim 1 further comprising a liquid cosmetic and/or nutraceutical preparation disposed within the fluid delivery conduit and the chamber.

17. The device of claim 1, wherein the hair flow passage is generally keyhole-shaped in an aspect perpendicular to the stems and the skin contact surfaces.

18. The device of claim 1, wherein the ratio of the distance between adjacent stems proximal to the skin contact surface to the maximum distance between adjacent stems is from about 0.34 to about 0.51.

19. The device of claim 1, wherein the input end is sealed with a removable cap.

20. The device of claim 1, wherein the manifold body further comprises proximal to the input end a removable cap comprising a pressure-equalization valve, and the chamber is closed at the input end.

21. A method of application of a fluid to skin bearing hair, the method comprising:
applying skin contact surfaces of the device of claim 1 to a first area of skin and/or hair of a human or animal subject;
moving the device in one of either of the directions of plow tips of the device for a first period of time while maintaining contact between the skin contact surfaces and a second area of the skin and/or hair; and
removing the skin contact surfaces from the skin and/or hair,
wherein at least some of the fluid is deposited as a layer on some or all of the second area of the skin and/or hair.
